(19) United States
(12) Patent Application Publication
(10) Pub. No.: US 2013/0012473 A1
(43) Pub. Date: Jan. 10, 2013

(54) COMBINED USE OF PHOSPHOLIPID AND SULFATE GROUPS-CARRYING POLYSACCHARIDES FOR INHIBITING METASTATIC SPREAD

(75) Inventors: Ulrich Massing, Freiburg (DE); Peter Jantscheff, Reinach (CH); Gerd Bendas, Bonn (DE); Martin Schlesinger, Bruhl (DE)

(73) Assignee: KTB TUMORFORSCHUNGSGESELLSCHAFT MBH, Freiburg (DE)

(21) Appl. No.: 13/635,465

(22) PCT Filed: Mar. 17, 2011
(86) PCT No.: PCT/EP2011/054032

§ 371 Date: Sep. 17, 2012

(30) Foreign Application Priority Data
Mar. 17, 2010 (EP) 10156776.6

(51) Int. Cl. A61K 31/737; A61P 35/04; A61K 31/727

(52) U.S. Cl. 514/56; 514/54

(57) ABSTRACT

The invention relates to a medicament comprising a phospholipid component and a polysaccharide component having polysaccharides containing sulfate groups for inhibiting metastatic spread of tumors. The two components may be in same or separate preparation.

The invention relates to a medicament comprising a phospholipid component and a polysaccharide component having polysaccharides containing sulfate groups for use in inhibiting metastatic spread of tumors. The two components may be in same or separate preparation.

### BACKGROUND OF THE INVENTION

Metastatic spread of solid tumors is a leading cause of death in the course of malignant diseases (Vantyghem, S. A. et al., Cancer Res. 65:3396-3403 (2005); Roepman, P. et al., Cancer Res. 66:11110-11114 (2006); Fokas, E. et al., Cancer Metastasis Rev. 26:705-715 (2007)). The underlying mechanisms of metastasis, such as tumor cell invasion into the blood system, interaction with blood components, adhesion to endothelial cells, and extravasation are highly complex and not yet completely understood (Joyce, J. A. and Pollard, J. W., Nat. Rev. Cancer 9:239-252 (2009); Klein, C. A., Nat. Rev. Cancer 9:302-312 (2009); Murdoch, C., Nat. Rev. Cancer 8:618-631 (2008); Nguyen, D. X. et al., Nat. Rev. Cancer 9:274-284 (2009); Psaila, B. and Lyden, D., Nat. Rev. Cancer 9:285-293 (2009); Smith, S. C. and Theodorescu, D., Nat. Rev. Cancer 9:253-264 (2009)). Consequently, there is presently no therapeutic approach to interfere with the metastatic spreading of tumors.

Recently, we observed a pronounced anti-metastatic effect of applying empty liposomes consisting mainly of hydrogenated phosphatidylcholine in an orthotopic human AsPC1 pancreatic cancer nude mouse model (Graeser, R. et al., Pancreas 38:330-337 (2009)). Since these phospholipids (PL)—in contrast to physiologically occurring PL—contained only saturated fatty acids, hydrogenated PL were supposed to be a key factor for this surprising effect (Graeser, R. et al., Pancreas 38:330-337 (2009)).

Physiologically, most plasma PL are associated with lipoproteins, surrounding the lipophilic inner core of triglycerides and cholesterolesters (Mead J. F. et al., e. Lipids V. Chemistry, Biochemistry and Nutrition. New York, NY: Plenum Press (1986)) and contain prominent amounts of unsaturated fatty acids (Phillips, G. B. and Dodge, J. T., J. Lipid Res. 8:676-681 (1967); Antar, M. A. et al., Biochem. J. 105:117-119 (1967)). Cells have access to these PL when hydrolysis of one fatty acid residue takes place, generating lysophospholipids (LysoPL) which can easily be taken up by the cells. PL hydrolysis occurs via enzymatic activity of either type II phosphor-lipases A2 (PLA2) (Kashiwagi, M. et al., Gut 45:605-612 (1999)), lecithin-cholesterol-acyl-transferase (LCAT; PLA2-activity) (Glomset, J. A., J. Lipid Res. 9:155-167 (1968)), or endothelial lipases (EL; PLA1-activity) (Chen, S. and Subbaiah, P. V., Biochim. Biophys. Acta 1771:1319-1328 (2007); Aoki, J. et al., Biochimie 89:197-204 (2007)). The hydrolysis of lipoproteins contributes to a pool of LysoPL, which represents about one tenth of the total amount of plasma PL (Mead J. F. et al., e. Lipids V. Chemistry, Biochemistry and Nutrition. New York, NY: Plenum Press (1986)). The concentration of LysoPC was found to be constant in blood plasma of healthy persons, usually ranging from 200 to 300 μM (Raffelt, K. et al., NMR Biomed 13:8-13 (2000); Takatera, A. et al., J. Chromatogr. B Analyt. Technol. Biomed. Life Sci. 838:31-36 (2006)) or 300 to 400 μM (Kuliszkiewicz-Janus, M. et al., Biochim. Biophys. Acta 1737:11-15 (2005), Sullentrop, F. et al., NMR Biomed. 15:60-68 (2002)), suggesting a sensitive balance of generation and removal of LysoPC, e.g. by cellular uptake. The fatty acid composition of plasma LysoPC in healthy individuals is comparable to that of the total plasma PL, showing that fatty acid hydrolysis takes place at the sn-1 and sn-2-position of the PL (Antar, M. A. et al., Biochem. J. 105:117-119 (1967); Kashiwagi, M. et al., Gut 45:605-612 (1999); Glomset, J. A., J. Lipid Res. 9:155-167 (1968); Chen, S. and Subbaiah, P. V., Biochim. Biophys. Acta 1771:1319-1328 (2007); Aoki, J. et al., Biochimie 89:197-204 (2007); Raffelt, K. et al., NMR Biomed 13:8-13 (2000)). Thus, assuming a similar enzymatic PL hydrolysis in liposomes, the anti-metastatic effect of empty liposomes as found in the AsPC1 pancreatic cancer model might be related to the increase of LysoPC containing saturated fatty acids.

This hypothesis is supported by the finding that aggressively growing prostate, pancreatic, and renal cancer cell lines—in contrast to blood-derived cells and cell lines—eliminate very high amounts of exogenous LysoPC in vitro, corresponding to up to 125 times their whole cellular PC content within 24 hours (Jantscheff et al. manuscript in preparation). Supplying LysoPC containing saturated fatty acids to these tumor cells induced a remarkable change in their PL fatty acid composition towards high ratios of saturated fatty acids. These findings are in line with the clinical observations that LysoPC levels are often decreased in advanced cancer patients (Kuliszkiewicz-Janus, M. et al., Biochim. Biophys. Acta 1737:11-15 (2005); Sullentrop, F. et al., NMR Biomed. 15:60-68 (2002); Kriat, M., et al., J. Lipid Res. 34:1009-1019 (1993); Kuliszkiewicz-Janus, M. et al., Anticancer Res. 16:1587-1594 (1996)), especially when weight loss and inflammatory processes occur (Taylor, L. A. et al., Lipids Health Dis. 6:17 (2007)).

The present invention is based on a study investigating whether the strikingly high consumption of LysoPC has an influence on the metastatic behavior of tumor cells. Therefore, we selected the highly metastatic melanoma cell line B16.F10 (Ludwig, R. J. et al, Cancer Res. 64:2743-2750 (2004); Laubli, H. et al., Cancer Res. 66:1536-1542 (2006); Fritzsche, J. et al., Thromb. Haemost. 100:1166-1175 (2008); Niers, T. M. et al., Clin. Exp. Metastasis 26:171-178 (2009)), investigated uptake and fate of the LysoPC and analyzed the impact on metastatic properties in an experimental metastasis model in mice. The metastasis of B16.F10 cells critically depends on their P-selectin binding ability to mediate interactions with platelets and to establish contact with the endothelium (Ludwig, R. J. et al, Cancer Res. 64:2743-2750 (2004)). Furthermore, the integrin VLA-4 on the surface of B16.F10 cells is also described as important factor to support endothelial binding to VCAM-1 (Fritzsche, J. et al., Thromb. Haemost. 100:1166-1175 (2008)). Therefore, we focused our study on changes in adhesion receptor activity after LysoPC treatment in vitro and investigated the correlation of the finding with the metastatic behavior of B16.F10 cells in mice.

The study with B16.F10 cells showed that LysoPC treatment of the cells was accompanied by an extremely fast removal of exogenous saturated LysoPC from medium and radical shifts in cell membrane fatty acid composition towards saturated fatty acids. The treatment had also a strong impact on adhesion receptor activity. VLA-4 mediated binding of B16.F10 cells to VCAM-1 as well as P-selectin dependent interaction with activated platelets were strikingly reduced, although their expression levels were not altered. These effects comparable to those observed with heparin, which is also known to inhibit VLA-4 binding to VCAM-1 and P-selectin binding to its ligand.

These findings were reflected in an intravenous metastatic mouse model using luciferase transduced B16.F10 cells. Repeated ex-vivo treatment of cells with LysoPC (6 times) resulted in significantly reduced metastatic lesions in the lung (51.7%, p=0.006). Similar reduction of metastasis with non-lysoPC-treated B16.F10 melanoma cells (43.6%, p<0.001) resulted from prior intravenous application of polysulfated polysaccharide Heparin (50 IU) into the mice. Since the anti-metastatic effect of heparin is also strongly caused—beside its anti-coagulant action—by blocking of the “VLA-4-VCAM-1” and “P-selectin—P-selectin ligand” but not “L-selectin—L-selectin ligand” interactions (Borsig, L., et al., Proc Natl Acad Sci USA, 99:2193-2198, (2002) and Laubli, H., et al., Cancer Res, 66:1536-1542 (2006)) the similar anti-metastatic effects (p=0.603) were not very surprising but consistent with the in vitro results described before (see above).

In the light of these findings, however, it was fully unexpected, that prior intravenous application of 50 IU (i.v.) unfractionated heparin followed by the application of repeatedly 450 μM LysoPC treated B16.F10 cells resulted in a strikingly increased reduction of metastases (18.4%, p=0.043).

In order to investigate how LysoPC treatment of the B16.F10 cells synergistically supports the inhibition of VLA-4- and P-selectin mediated adhesion of the cells, surface changes of the LysoPC treated cells were investigated by scanning electron microscopy. First studies showed remarkable morphological surface changes of the LysoPC treated cell, so that a sufficient accessibility of the adhesion molecules might not longer possible, which could be one explanation oft the synergistic activity of heparin and LysoPC. In conclusion, it was shown for the first time that hydrogenated LysoPC can inhibit the hematogeneous dissemination of B16.F10 melanoma cells, probably due to reduced cell adhesion via VLA-4 and P-selectin. The mechanisms, however, seem to be partly different from heparin effects since the two agents act synergistically.

### SUMMARY OF THE INVENTION

The invention thus relates to

(1) a medicament for use in treating or preventing metastatic spread of tumors comprising a phospholipid component and a polysaccharide component having polysaccharides containing sulfate groups (hereinafter shortly referred to as “polysaccharide component”);

(2) a preferred embodiment of aspect (1) above, wherein said phospholipid component comprises at least one phospholipid compound represented by the formula I

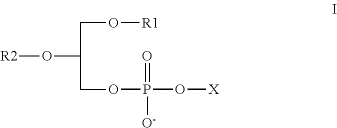

wherein

R¹ and R² are independently selected from H and C₁₆₋₂₄ acyl residues, which may be saturated or unsaturated and may carry 1 to 3 residues R³ and wherein one or more of the C-atoms may be substituted by O or NR⁴;

X is selected from H, —(CH₂)ₙ—N(R⁴)₃⁺, —(CH₂)ₙ—CH(N(R⁴)₃⁺)—COO⁻, —(CH₂)ₙ—CH(OH)—CH₂OH and —CH₂(CHOH)ₙ—CH₂OH, wherein n is an integer from 1 to 5;

R³ is independently selected from H, lower alkyl, F, Cl, CN und OH; and

R⁴ is independently selected from H, CH₃ und CH₂CH₃,

or a pharmacologically acceptable salt thereof;

(3) a preferred embodiment of aspect (1) or (2) above, wherein the polysaccharide component comprises at least one component selected from heparins such as fractionated heparins, non-fractionated heparins, anti-coagulating heparins, non-anti-coagulating heparins, artificial heparins;

(4) a phospholipid component and a polysaccharide component as defined in any one of (1) to (3) above for (combined) use in inhibiting metastatic spread of tumors; and

(5) a method for inhibiting metastatic spread of tumors, which comprises contemporaneously or subsequently administering a patient in need of such treatment a phospholipid component and a polysaccharide component as defined in any one of (1) to (3) above.

### SHORT DESCRIPTION OF THE FIGURES

FIG. 1: Removal of exogenous LysoPC by B16.F10 melanoma cells. (A) LysoPC removal kinetics in non-transduced or luciferase-transduced B16.F10 melanoma cell lines (mean values from triplicates of three independent experiments) were determined by a commercial PC/lyso-PC assay and corrected by the released free choline (see Materials and Methods, FIG. 7). A very rapid degradation/consumption of LysoPC (24 h), or a total elimination at least 48 h after adding are evident. (B) Repeated feeding (every 48 h) with 450 μM LysoPC (C17:0) did not affect LysoPC degradation kinetics, LysoPC consumption as determined by 4 repeated additions of LysoPC for at least 9 d.

FIG. 2: Influence of exogenous LysoPC treatment on B16.F10 cell binding to VCAM-1. (A) Adhesion of activated B16.F10 cells (▪non activated cells) to immobilized VCAM-1 was determined in a microscopic flow chamber assay. While 72 h incubation with 450 μM LysoPC significantly (p<0.001) reduced the cell binding ability to VCAM-1, 300 μM LysoPC had only minor effects. (B) Prolonging incubation to 13 d and adding exogenous LysoPC every second day augmented the effects of 450 μM LysoPC (p<0.001), but not of 300 μM LysoPC.

FIG. 3: Impact of exogenous LysoPC on the surface expression of VLA-4 or VCAM-1- and P-selectin-ligands. (A) Surface expression of VLA-4 on B16.F10 cells treated with 450 μM LysoPC for 13 days was controlled by flow cytometric analysis. FACS analysis using a rat anti-mouse VLA-4 specific mAb showed that VLA-4 expression on B16.F10 cells was not changed by the LysoPC treatment. (B) FACS histograms of functional binding capacity of VLA-4 on LysoPC treated B16.F10 cells, on the contrary, show a slightly reduced binding shift of soluble VCAM-1 Fc chimera. (C) FACS binding analysis of soluble P-selectin Fc chimera on 450 μM LysoPC treated B16.F10 cells clearly showed that the expression and accessibility of P-Selectin ligands were not modified by the treatment. FACS histograms (A-C) show one representative of three independent experiments. In all experiments cells only incubated with the respective FITC-conjugated detection antibody served as control.

FIG. 4: Fatty acid composition and morphology of LysoPC treated B16.F10 ELN cells. Composition of fatty acids (% of total FA) in cell lysates was determined after feeding B16.F10 cells with 450 μM HEP-LysoPC (A) after different times by GC analysis. Composition of membrane fatty acids was not further enhanced by repeated (1-4 times) addition of exogenous LysoPC (B) every 48 h (+/ 48/0 h) and remained nearly unchanged (only shown C17:0) also after discontinuing LysoPC application (C) after 48 h by control medium (+/+48 h).

FIG. 5: Impact of LysoPC pre-treatment on metastatic spread in vivo. Metastatic spread of B16.F10 ELN melanoma cells into the lung of syngeneic male C57.B16 mice was quantified using an in vitro luciferase assay (day 14). As shown in the dot plot diagram (n=number of animals), long-term (450 μM—six times) but not short-term (300 μM—two times) LysoPC treatment resulted in significant (p=0.006) reduced lung invasion. Mean percentages were calculated for each group relative to untreated controls (100%). Representative macroscopic images confirm these results (lower part) with decreased numbers of visible metastatic foci in the 450 μM LysoPC group. In one case (marked by Δ* in upper part) of lung homogenates (mice 1179/09) from long-term LysoPC group, displaying extremely high luciferase activity (RLU: 4.694.952 compared to a mean of 1.698.601) no augmented metastatic infiltration was visible in the lung (lower part: 1179*).

FIG. 6: Effect of LysoPC pre-treatment and heparin injection on metastatic spread of B16.F10 melanoma cells. Comparison of the significant anti-metastatic effects (compared to untreated cells) of either, a long-term (six times 450 μM) LysoPC treatment (p=0.006) of B16.F10 ELN cells, or of a pre-treatment of C57/B16 mice with 50 IU unfractionated heparin 30 min before injection (p=0.004) of untreated B16.F10 ELN melanoma cells (Heparin) or the combination of the two treatment regimes (p<0.001). Columns represent mean values and standard deviations of RLU (n=number of animals). Mean percentages were calculated for each group and related to untreated controls (100%). No difference was seen (p=0.603) between the LysoPC and the heparin groups.

FIG. 7 shows the following: (A) Effect of bovine serum albumine (BSA) on LysoPC-mediated toxicity; (B), (C) and (D) Colorimetric assays for the quantification of LysoPC removal from the supernatant of B16.F10.

FIG. 8 shows the impact of short-term exogenous LysoPC treatment of B16.F10 cells on P-selectin-mediated platelet binding, morphology and migratory properties on a fibronectin substrate. (A) Motility data of B16.F10 cells on fibronectin layers derived by SACED assay displayed significant (p<0.05) differences in migration velocities. (a) B16.F10 cells incubated with 450 μM LysoPC for 24 h showed a slower lamellipodia retraction velocity than cells without LysoPC. (b) Analysis of lamellipodia dynamics (i.e. frequencies) revealed attenuated dynamics (p<0.05) of lamellipodia protrusion and retraction regarding the cells incubated with 450 μM LysoPC for 24 h. (B) Binding of calcein-labeled TRAP-14 activated platelets to B16.F10 cells treated with 450 μM LysoPC for 48 h was analyzed by fluorescence microscopy. (C) Cell surface alterations with respect to LysoPC treatment of B16.F10 cells were determined by scanning electron microscopy (SEM).

FIG. 9 shows the detection of metastatic lesions by luciferase-transduced B16.F10 melanoma cells in the lung and other tissues. As shown in A) significant metastasis could be detected first at day 7 and reached a plateau at day 14 in the lung. As shown in (B) (day 14) and (C) (day 19) metastatic spread was also observed in the other tissues of individual mice, but to an at least 1,000 times lesser extent than in the lung. (D) Overlays of in vivo images and mouse photographs allowed to localize the metastatic lesions in lung and at the injection sites ((D): black arrows) even in late images ((D): day 14).

### DETAILED DESCRIPTION OF THE INVENTION

Aspects (1) to (3) of the invention pertain to a medicament for use in treating or preventing metastatic spread of tumors (hereinafter shortly referred to as “medicament of the invention”) comprising a phospholipid component and a coagulation modulating component.

In the medicament of aspect (1) it is preferred that the phospholipid component comprises at least one phospholipid containing ω-3-fatty acids and/or containing no or only low amounts of ω-6 fatty acids. In a more preferred embodiment said at least one phospholipids contains no or only low amounts of ω-6 fatty acids, preferably no or only low amounts of arachidonic acid, and/or said at least one phospholipids contains ω-3-fatty acids, preferably eicosapentaenoic acid (EPA) or docosahexaenoic acid (DHA).

It is further preferred that in the phospholipid component said at least one phospholipid may be a 1,2-diacylglycerophospholipid, 1-acylglycerophospholipid or 2-acylglycerophospholipid having saturated and/or unsaturated acyl residues or pharmacologically acceptable salts thereof.

Further, in the phospholipid component said at least one phospholipid may contain acyl residues selected from saturated acyl residues, ω-3- and ω-9-fatty acid residues and mixtures thereof, and the content of said acyl residues preferably is at least 50% by weight of the total acyl residues present within the phospholipid component. Still further, in the phospholipid component said at least one phospholipid may be a phosphatidylcholin. Even further, the phospholipid component may further contain triglycerides, free fatty acids diglycerides and/or monoglycerides, wherein the total content of said further glycerides and fatty acids is no more than 90% by weight of the total lipids of the component. Even further, the phospholipid component may contain oils, preferably fish oils. Even further, if the phospholipid component comprises a phospholipid containing ω-3-fatty acids, the phospholipid component may further comprises an oil having a high content of ω-3-fatty acids. Even further, in the phospholipid component said at least one phospholipid may be a phospholipid of marine origin (MPL), preferably an MPL derived from water animals such as fish, and most preferably an MPL isolated from fish liver or roe. Even further, in the phospholipid component said at least one phospholipid may be a hydrogenated phospholipid containing essentially saturated fatty acids, such as palmitic acid (hexadecaoic acid), heptadecanoic acid, stearic acid. Even further, in the phospholipid component said at least one phospholipid is a milk derived phospholipid having no or only low amounts of ω-6 fatty acids, such as arachidonic acid, and containing high amounts of ω-9 fatty acids, such as oleic acid. According to aspect (2) of the invention the phospholipid component comprises at least one phospholipid compound represented by the formula I

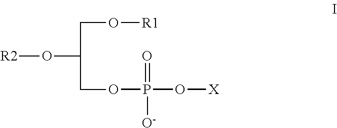

wherein R¹ and R² are independently selected from H and C₁₆-₂₄ acyl residues, which may be saturated or unsaturated and may carry 1 to 3 residues R³ and wherein one or more of the C-atoms may be substituted by O or NR⁴; X is selected from H, —(CH₂)ₙ—N(R⁴)₃⁺, —(CH₂)ₙ—CH(N(R⁴)₃⁺)—COO, —(CH₂)ₙ—CH(OH)—CH₂OH and —CH₂(CHOH)ₙ—CH₂OH, wherein n is an integer from 1 to 5; R³ is independently selected from H, lower alkyl, F, Cl, CN and OH; and R⁴ is independently selected from H, CH₃ and CH₂CH₃, or a pharmacologically acceptable salt thereof.

Suitable lower alkyls for R³ include straight, branched or cyclic alkyls having 1 to 6 carbon atoms which may be saturated or unsaturated, among which saturated alkyls having 1 to 3 carbon atoms (notably methyl, ethyl, n-propyl, isopropyl and cyclopropyl) are particularly preferred. It is preferred R¹ and R² are independently selected from H and unsubstituted C₁₆₋₂₄ acyl residues, which may be saturated or unsaturated, and X is selected from a choline, serine, ethanolamine, glycerol and inositol residue. Even more preferred is that at least one of R¹ and R² is an ω-3-fatty acid residue having at least 20 C-atoms such as an eicosapentaenoic acid (EPA) or docosahexaenoic acid (DHA) residue or is an ω-9-fatty acid residue having at least 18 C-atoms such as an oleic acid, erucic acid or nervonic acid residue.

Further preferred is that the content of ω-3- and ω-9-fatty acid residues is at least 20%, more preferably at least 35%, most preferably at least 45% by weight of all acyl residues of the phospholipid component. Even further preferred is that the weight ratio of ω-3-fatty acids and/or ω-9-fatty acids to ω-6-fatty acids in the acyl residues of the phospholipid component is at least 10:1, most preferably at least 15:1. Still further preferred is when R¹ and R² are independently selected from H and unsubstituted and saturated C₁₆₋₂₄ acyl residues, such as palmitoyl, heptadecanoyl and stearoyl. Still further preferred is when R¹ and R² are independently selected from H and unsubstituted ω-9-C₁₆₋₂₄ alkenoyl residues, most preferred are oleoyl residues.

In a particular preferred embodiment of aspects (1) and (2) the at least one phospholipid is selected from oleoyl-glycerophosphocholine, hexadecanoyl (i.e. palmitoyl)-glycerophosphocholine, heptadecanoyl-glycerophosphocholine and stearoyl-glycerophosphocholine and the respective lyso compounds, in particular 1-heptadecanoyl-2-hydroxy-sn-glycero-3-phosphocholine, 1-hexadecanoyl-sn-glycero-3-phosphocholine and stearoyl-sn-glycero-3-phosphocholine.

According to aspect (2) of the invention the polysaccharid component comprises at least one polysaccharide containing sulfate groups, such polysaccharide preferably is a compound selected from heparins such as fractionated heparins (including Tinzaparin (Innohep)—LEO, Enoxaparin (Clexane)—Sanofi Aventis, Nadroparin (Fraxiparin)—GSK, Dalteparin (Fragmin)—Pharmacia and Certoparin (Mono-Embolex-Novartis, non-fractionated heparins (including UFH Calcium (Calciparin)—Sanofi Synthelabo and UFH—Ratiopharm), anti-coagulating heparins, non-anti-coagulating heparins and artificial heparins. Fondaparinux is no suitable heparin within the meaning of the invention. Particular preferred polysaccharides of the invention are fractionated heparins.

In aspects (1) to (3) of the invention the phospholipid component and the polysaccharide component may be present in two separate preparations. Here it is preferred that the phospholipid component is suitable to be administered orally, intravenously, subcutaneously, intracutaneously, intraperitonally or rectally, and the polysaccharide component is suitable to be administered intravenously, subcutaneously, intracutaneously or intraperitonally.

In another embodiment the phospholipid component and the polysaccharid component may be present in one single preparation, preferably said preparation is a heparinized liposome and/or is suitable to be administered intravenously, subcutaneously, intracutaneously or intraperitonally. The medicament of the invention is particularly suitable for inhibiting metastatic spread of solid tumors, preferably for treating or preventing metastatic spread of melanoma, pancreascarzinoma, prostatacarzinoma, ovarialcarzinoma, and mammacarzinoma. In case of intravenous, subcutaneous, intracutaneous or intraperitonal application of the medicament the phospholipid component preferably comprises lysophospholipids, i.e. compounds of formula (I) wherein R¹ or R² is H.

One of the pivotal steps of metastases (Teoh, M. L. et al., Cancer Res. 69:6355-6363 (2009) is the hematogenous dissemination of tumor cells (Ludwig, R. J. et al, Cancer Res. 64:2743-2750 (2004); Laubli, H. et al., Cancer Res. 66:1536-1542 (2006); Al-Mehdi, A. B. et al., Nat. Med. 6:100-102 (2000); Dimitroff, C. J. et al., Cancer Res. 64:5261-5269 (2004); Glinsky, V. V. et al., Cancer Res. 63:3805-3811 (2003), Thomas, S. N. et al., J. Biol. Chem. 283:15647-15655 (2008)), which includes the i) intravasation of tumor cells, ii) their survival in the blood stream, iii) tumor cell binding to vascular endothelial cells, and iv) the extravasation into target tissue(s). Cellular adhesion receptors, i.e. P- and L-selectin, and VLA-4 are considered as key players in the metastatic process, mediating tumor cell aggregation with platelets or leukocytes (Honn, K. V. et al., Cancer Metastasis Rev. 11:325-351 (1992); Karpatkin, S. and Pearlstein, E., Ann. Intern. Med. 95:636-641 (1981)) resulting in tumor cell emboli that physically come to rest in the microvasculature (Laubli, H. et al., Cancer Res. 66:1536-1542 (2006)).

The present invention is based on the recently observed phenomenon that empty liposomes consisting of saturated PL applied in an orthotopic human AsPC1 pancreatic cancer nude mouse model displayed pronounced anti-metastatic effects (Graeser, R. et al., Pancreas 38:330-337 (2009)). Given the clinical observation that advanced metastatic cancer patients display significantly decreased LysoPC levels (Kuliszkiewicz-Janus, M. et al., Biochim. Biophys. Acta 1737:11-15 (2005); Sullentrop, F. et al., NMR Biomed. 15:60-68 (2002), Kriat, M., et al., J. Lipid Res. 34:1009-1019 (1993), Kuliszkiewicz-Janus, M. et al., Anticancer Res. 16:1587-1594 (1996)), saturated LysoPC as degradation product from the liposomes were postulated as the pivotal agents for anti-metastatic activity of empty liposomes. To revise this hypothesis, the LysoPC metabolism was investigated in B16.F10 melanoma cells that were exemplarily selected for solid tumors with high metastatic activity.

It could be shown for the first time that LysoPC, added to B16.F10 melanoma cells in physiologically relevant concentration (300 μM-450 μM), is rapidly removed from the supernatant in vitro. The removed amount of LysoPC corresponds to approximately 125 times the total amount of cellular PLs. This effect, which was not saturable, has to be discussed as one reason for the strongly reduced LysoPC amounts in patients with advanced cancer suffering from severe tumor related weight loss. Of interest in this context is the finding that the oral application of PLs (from salmon roe) stabilizes the weight of affected patients, as shown in a recent clinical study (Taylor, L. A. et al., phospholipids-a promising new dietary approach to tumor-associated weight loss. Support Care Cancer (2009)),

Investigating the fate of the removed LysoPC, the comparable increase of free fatty acids in the supernatant probably should result from either LysoPC or membrane PL degradation (Jantscheff et al. manuscript in preparation). It is not clear whether this degradation occurs after the uptake of LysoPC into the cell membranes. Besides degradation, a part of the LysoPC was used by the tumor cells for PL synthesis during membrane turnover and cell growth: Supplying LysoPC with C16- and C17-fatty acid residues induced a dramatic increase in the percentage of these fatty acids in the cellular PLs.

Furthermore, the treatment of the B16.F10 cells with C16- or C17-LysoPC at concentrations corresponding to physiological human plasma levels has a strong impact on the tumor cell behavior with respect to adhesion receptors as well as metastases formation in vivo. The decreased functionality of the integrin VLA-4 and P-selectin binding in vitro correlates impressively with the inhibitory effects of the LysoPC cell pre-treatment in an experimental metastasis model in mice.

However, the molecular mechanisms of the massive change in FA-composition after LysoPC incubation leading to reduced adhesion receptor activity could not fully be elucidated in this study. On one hand, the balance between saturated and unsaturated fatty acids is a fundamental biophysical determinant of membrane fluidity (Mansilla, M. C. et al., Subcell. Biochem. 49:71-99 (2008)). The so-called homeoviscous adaptation is regulated in a complex mode, influencing important membrane properties as flexibility, lipid raft composition, or permeability (Mansilla, M. C. et al., Subcell. Biochem. 49:71-99 (2008), Stulnig, T. M., et al., J. Biol. Chem. 276:37335-37340 (2001); Callaghan, R. et al., Biochim. Biophys. Acta 1175:277-282 (1993); Hac-Wydro, K. and Wydro, P., Chem. Phys. Lipids 150:66-81 (2007)). These effects might induce changes in receptor interaction and consequently signaling processes.

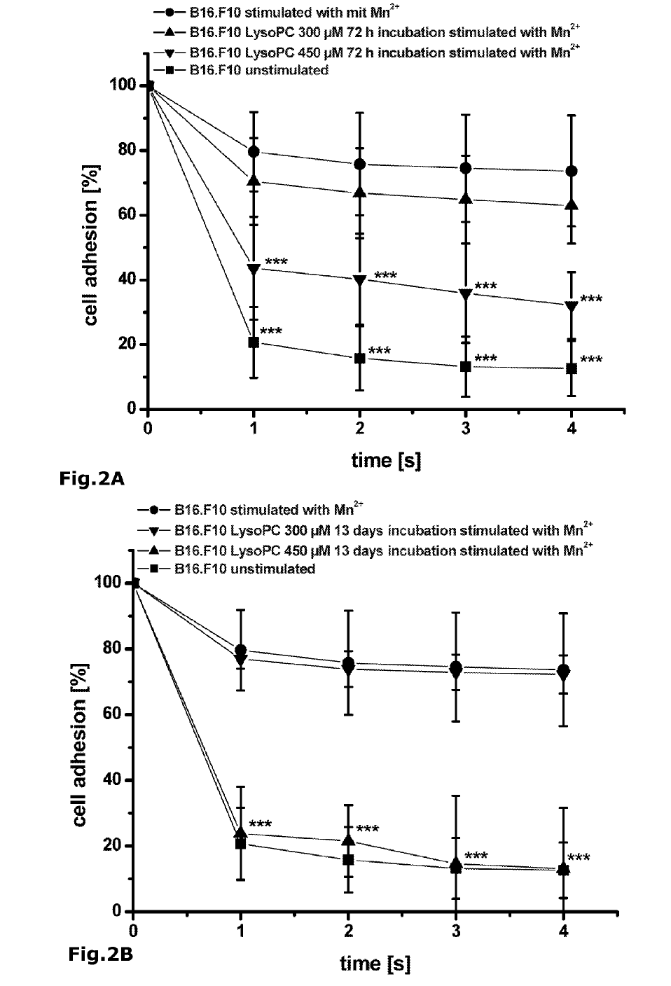

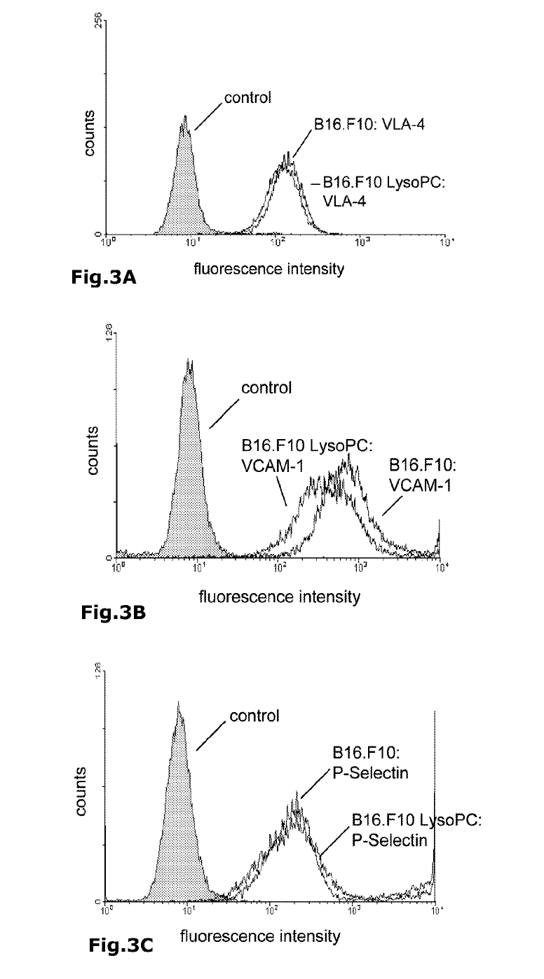

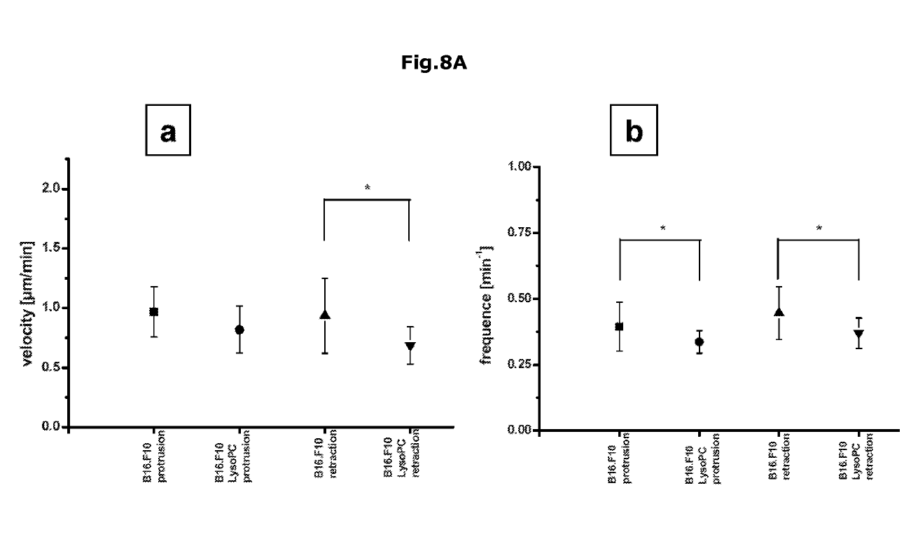

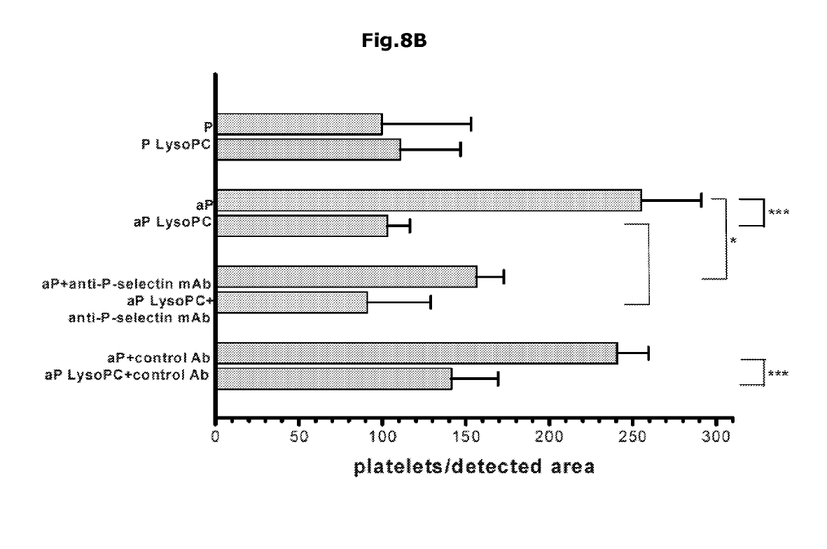

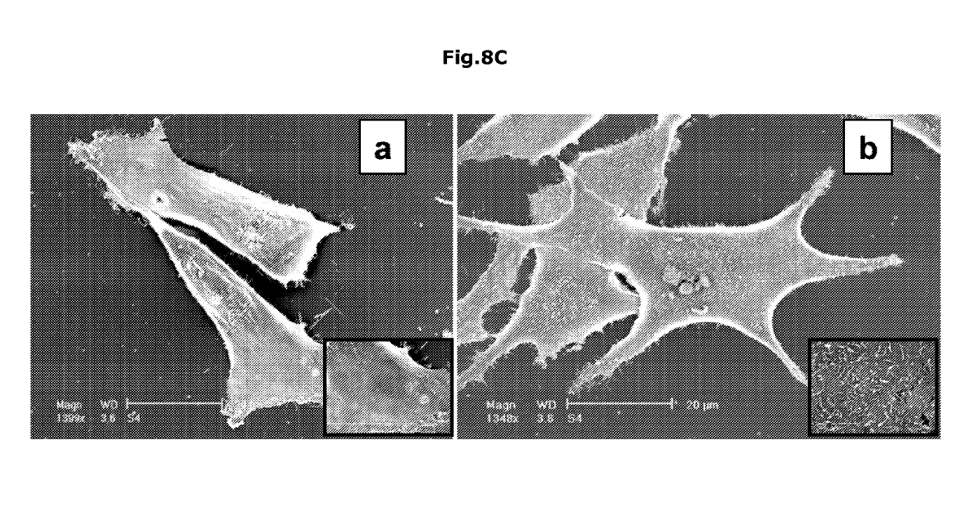

On the other hand, morphological changes of the cell membrane could be the reason for altered functionality of receptors. SEM data strongly support this assumption (FIG. 8C), since LysoPC incubation induced a dramatic increase in membrane protrusions covering the cell surface, while normal cells display a moderate number of lamellipodia at a rather smooth membrane surface. It can be assumed that these lamellipodia spatially restrict the accessibility of the receptors for cellular or support-fixed ligands, but to a lesser extent for soluble binding structures. This would explain the similar binding of VLA-4 antibodies, P-selectin- or VCAM-1 Fc chimera on LysoPC treated and untreated cells (FIG. 3A, B, C), in contrast to prevention of cell adhesion onto immobilized ligands (FIG. 2A, B) as well as impaired interaction with platelets (FIG. 8 B) of the LysoPC treated cells. These morphological changes occur rapidly and seem to be accomplished within a three day period, so we presently cannot explain the strong dependence of effects on the LysoPC concentration (300 μM vs. 450 μM) and the further decrease in cell adhesion with even longer LysoPC incubation times (FIGS. 2A and B). This also applies for the rapid change in membrane lipid composition, which is not directly reflected in functional changes. But this increase in lamellipodia furthermore reduced the ability of the cells to migrate on fibronectin, which may be regarded as model for reduced tissue transmigration and thus, as further explanation for reduced metastatic activity.

The in vitro findings on reduced adhesive and migratory characteristics of the B16.F10 cells after LysoPC treatment were fully reflected by the in vivo data, with special respect to the concentration threshold of 450 μM LysoPC. The establishment and application of the luciferase-transduced B16.F10 ELN cells for these experimental metastatic assays in mice strongly increased the informational value and allow a more sensitive analysis, in contrast to the visual evaluation of the metastatic foci in the lungs. These data imply that, referring to the anti-metastatic effects of empty liposomes (Graeser, R. et al., Pancreas 38:330-337 (2009)), the degradation of hydrogenated PL from the empty liposomes increases the level of hydrogenated LysoPC, which possesses anti-metastatic activities via reduced adhesion receptor activity. Thus, exogenous LysoPC should partially act at the same target as heparin. This is supported by the findings that both, 450 μM LysoPC or heparin at an established concentration resulted in comparable levels of decreased metastasis. However, this can not simply explain the synergistic effect of both treatment regimes in reducing metastasis. Assuming from our in vitro findings, a 450 μM LysoPC treatment has a maximum effect on the reduction of P-selectin binding and VLA-4 activity. Since a dose of 50 IU heparin is also almost the maximum dose (60 IU) to inhibit metastatic spread in the B16.F10 model (Ludwig, R. J. et al., Cancer Res. 64:2743-2750 (2004)), a synergistic rather than additive mechanism(s) of the two substances is plausible.

The invention is further explained in the following non-limiting examples.

### EXAMPLES

Material and Methods

Antibodies and adhesion molecules: Recombinant mouse VCAM-1 Fc chimera, anti-mouse CD49d mAb (integrin α₄ chain), and recombinant mouse P-selectin Fc chimera were purchased from R&D Systems GmbH (Wiesbaden-Nordenstadt, Germany). Anti-human IgG (Fc specific)-FITC and human fibronectin were obtained from Sigma-Aldrich Chemie GmbH (Taufkirchen, Germany).

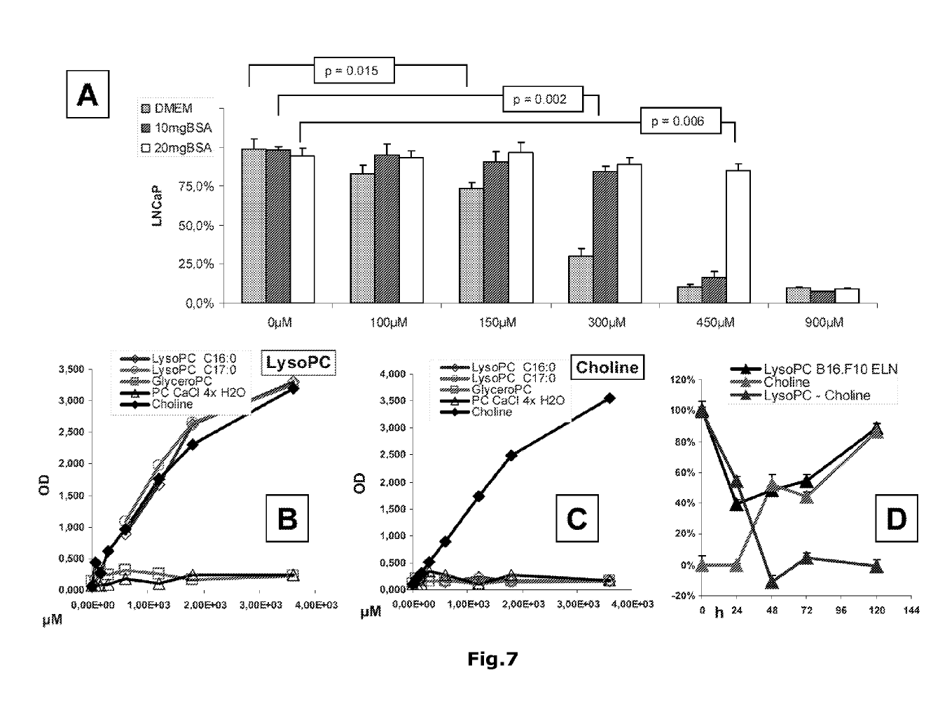

Cell culture and LysoPC feeding: Mouse melanoma cell line B16.F10 (ATCC, CRL-6475) was passaged in vitro in DMEM as described before (Fritzsche, J. et al., Thromb. Haemost. 100:1166-1175 (2008)). Cells were routinely (monthly) tested to be negative of mycoplasms, their morphological appearance observed by microscopic means and examined for the expression of adhesion receptors and ligands by flow cytometry. LysoPC removal (1-Heptadecanoyl-2-hydroxy-sn-glycero-3-phosphocholine, (C17:0; HEP-LysoPC), Avanti Polar Lipids, Inc., or 1-Hexadecanoyl-sn-glycero-3-phosphocholine, (C16:0; HEX-LysoPC), Sigma, Taufkirchen, Germany was determined using 1×10⁵ cells in 24-well tissue culture plates (Greiner BioOne, Frickenhausen, Germany). Non-cytotoxic LysoPC/BSA DMEM-solutions (FIG. 7 A) were prepared by adding 20-40 mg/ml bovine serum albumine (BSA, PAA Laboratories GmbH, Cölbe, Germany). Cell culture supernatants were collected from triplicates of LysoPC treated and untreated control wells. Cells were removed from tissue culture plates using trypsin/EDTA. Centrifuged supernatants and cell pellets were stored at −20° C. and −80° C. until analysis, respectively. Long-term treatments of cells were performed as mentioned above, adding and removing cells, LysoPC and supernatants as specified in the results section. Cells for in vivo application were cultivated in 250 ml culture flasks either with 15 ml of 450 μM or 300 μM LysoPC, or culture medium as control.

Generation of luciferase expressing B16.F10 melanoma cells: For sensitive detection of metastases in vivo in lung and other murine tissues, stably luciferase-transduced B16.F10 ELN melanoma cells were established as recently described (Bornmann, C. et al., Cancer Chemother. Pharmacol. 61:395-405 (2008)).

BrdU proliferation assay: Proliferation and viability were determined by BrdU and WST-1 assay (Roche Diagnostics GmbH, Penzberg, Germany) according to the manufacturer's instructions.

Determination of LysoPC concentration: LysoPC concentration was determined by an enzymatic PL (PC/LysoPC) assay containing phospholipase D and choline oxidase (mti diagnostics GmbH, Idstein, Germany). For the determination of free choline, we developed a similar assay which does not contain phospholipase D, and thus is specific for free choline (see Supplementary Content). GlyceroPC was determined using the assay for free choline adding 1 IU/ml of sn-glycerol-3-phosphocholine phosphodiesterase (Sigma, Taufkirchen, Germany). The specificity of the three assays was assured by analyzing different concentrations of LysoPC, free choline, glycerophosphocholine and PC (FIG. 7 B-D).

Determination of fatty acid composition in cells and culture medium: Lipid extraction was performed according to the modified method of Bligh and Dyer (Bligh, E. G. and Dyer, W. J., Can. J. Biochem. Physiol. 37:911-917 (1959). For derivatization, the dried lipid extracts were subjected to methanol and TMSH and heated up to 100° C. for 10 min (Techne Dri Block DB-3D Techne, Stone, UK). Gas chromatography analysis was performed by an HP 5890 Series II Plus equipped with autosampler GC/SFC injector and flame ionisation detector (FID) with helium as carrier gas at 1 ml/min. Oven temperature program started with 150° C. for 3 min, up to 220° C. with a rate of 5° C./min, 220° C. for 3.5 min, split injection (split flow 100 ml/min, split ratio 1:100), 5 μl of injection volume, injector temperature 260° C. and FID at 280° C. on an Agilent DB-23 column (30 m, 0.25 mm ID, 0.25 μm). Integration of peak areas was performed with the HP software HP GC ChemStation (Rev.A.06.03[509]) running the GC system. Identification of peaks was accomplished by comparison to a fatty acid standard mixture.

Platelet isolation: Platelets from blood of healthy volunteers were separated, routinely tested for viability, and activated by TRAP-14 (Bachem GmbH, Weil am Rhein, Germany) as recently described (Fritzsche, J. et al., Thromb. Haemost. 100:1166-1175 (2008)). Isolated platelets were stored at 37° C. in a water bath.

Flow cytometry analysis and interaction of B16.F10 cells with platelets: Interaction of murine recombinant P-selectin Fc chimera, murine VCAM-1 Fc chimera, and anti-mouse CD49d (integrin α₄ chain) mAb as well as of calcein-labeled platelets with B16.F10 melanoma cells with or without LysoPC incubation, was revealed according to recently described protocols (Fritzsche, J. et al., Thromb. Haemost. 100:1166-1175 (2008)).

Flow chamber assay: The interaction of B16.F10 cells with VCAM-1 Fc chimera under physiological flow conditions was analyzed on glass slides coated with VCAM-1 Fc chimera in a parallel plate flow chamber, as described in detail in our previous investigations (Fritzsche, J. et al., Thromb. Haemost. 100:1166-1175 (2008)).

Quantification of cell motility: Cell migration of B16.F10 cells cultivated on fibronectin (Sigma-Aldrich Chemie) covered dishes was determined with or without LysoPC. Phase contrast image series of motile cells were obtained 48 h later using an inverted microscope (Nikon, Dusseldorf, Germany) and an incubation chamber for constant temperature, applying NIS Elements AR 2.30 software and a camera (COOL-1300I; Vosskuhler GmbH, Germany)

Lamella dynamics and cell migration velocity were analyzed by the computer-assisted stroboscopic analysis of cell dynamics (SACED). At least 15 individual cells, incubated with or without LysoPC, were monitored over a period of 10 min by capturing digital images every 2 s. Subsequently, eight areas of interest were marked on each image by lines that crossed the cell lamella. The resulting 1 pixel wide areas were cut and lined up in time space plots that allowed the quantification of relevant motility data (Hinz, B. et al., Exp. Cell. Res. 251:234-243 (1999)).

Scanning electron microscopy (SEM): For SEM analysis, B16.F10 cells incubated with or without LysoPC were washed with PBS, fixed with 3% paraformaldehyde/1% glutaraldehyde in PBS for 30 min according to Svitkina et al. (Svitkina, T. M. et al., Eur. J. Cell. Biol. 34:64-74 (1984)). All specimens were mounted on aluminium sample holders and coated with a 2 nm layer of platinum/palladium in a HR 208 sputter coating device. Scanning EM was performed with a XL 30 SFEG (Philips, Eindhoven, Netherlands).

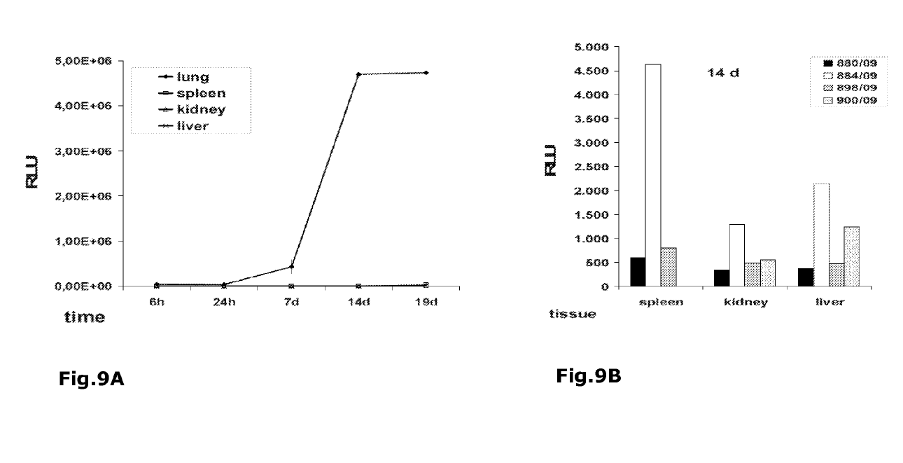

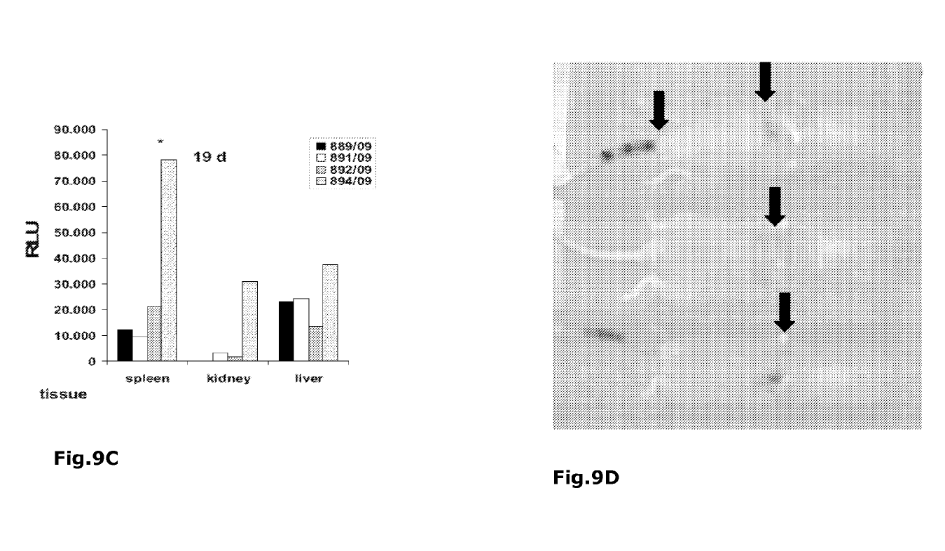

Animal experiments: Lung metastases were induced by injecting 3×10⁵ cells in 100 μl PBS into the tail vein of male C57BI/6N mice (Charles River Laboratories, Sulzfeld, Germany) (Ludwig, R. J. et al., Cancer Res. 64:2743-2750 (2004)). Animals received B16.F10 ELN cells either treated in vitro 7× with 450 μM LysoPC (long-term) within 10 days, 2× with 300 μM LysoPC within 2 days, or cultured in DMEM as control (identical frequency of medium change, cell splitting). In a further experiment, animals were pre-treated 30 min before cell injection with 50 IU (Ludwig, R. J. et al., Cancer Res. 64:2743-2750 (2004)) unfractionated heparin (Ratiopharm GmbH, Ulm, Germany), followed by injection of either long-term 450 μM LysoPC treated or DMEM control medium cultured B16.F10 ELN cells. Experiments were terminated on day 14 (see establishment of the model—FIG. 9). Tissues were removed and stored as snap frozen samples. Metastatic lesions were quantified by homogenizing the tissues in luciferase lysis buffer and measuring in a luciferase assay (Promega E1501) as described (Jantscheff, P. et al., Establishment of human orthotopic LNCaP prostate cancer xenograft metastases model: Effect of Gemcitabine and GemLip. Clin. Exp. Metastasis: in press (2009); Jantscheff, P. et al., Prostate 69:1151-1163 (2009)).

Statistics: Statistical analyses were performed using student's t-test or—if normality test failed—using Mann-Whitney Rank Sum Test (SigmaStat 3.1).

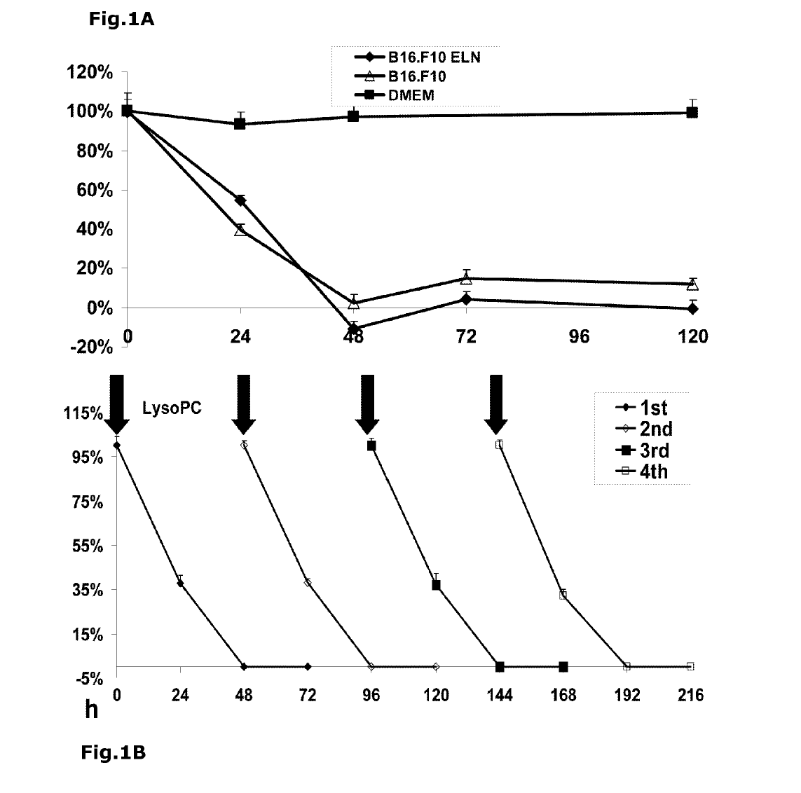

Example 1: Removal of exogeneous LysoPC from the medium by B16.F10 melanoma cells. Removal of exogenous LysoPC from the medium, i.e. consumption or degradation by the B16.F10 melanoma cells was measured in a time-course experiment in vitro (FIG. 1). In order to avoid cell lysis induced by physiological LysoPC concentrations (450 μM) BSA was added to the culture medium (FIG. 7 A). Both, B16.F10 as well as the luciferase transduced B16.F10 ELN cells caused a rapid reduction of the LysoPC, while LysoPC concentration was not changed in absence of the cells (FIG. 1A).

This striking removal of LysoPC was accompanied by an equimolar release of free fatty acids (not shown), as well as significant amounts of free choline (FIG. 7 D) and/or of GlyceroPC (not shown) into the culture supernatant. The rapid elimination of LysoPC by B16.F10 cells was not influenced by repeated (every 48 h) additions of exogenous LysoPC (FIG. 1B).

LysoPC removal was determined by measuring total choline (LysoPC and free choline) and subtracting the values of free choline as measured by a separate free choline assay (for details see FIG. 7 B-D). The rate of LysoPC reduction was found to be independent of the LysoPC-concentration (300 μM and 450 μM) or the type of LysoPC species (HEX-LysoPC or HEP-LysoPC) (not shown).

Example 2: Impact of LysoPC treatment of B16.F10 cells on adhesion receptor activity in vitro. In order to investigate the concentration and time dependent effects of LysoPC on the receptor mediated cell binding ability, VLA-4 adhesion of B16.F10 cells to a support-fixed VCAM-1 layer was investigated (FIG. 2A). While a single incubation step of cells with 450 μM LysoPC for 72 h before VLA-4 activation significantly impaired the cell binding, lower LysoPC concentration of 300 μM did not mediate significant binding decrease. The LysoPC effects on adhesion became amplified by repeated LysoPC feeding. B16.F10 cells treated six times with 450 μM LysoPC (13 days) showed complete binding inhibition and behaved like non-activated control cells, while repeated addition of 300 μM LysoPC had no effect in reducing the VLA-4 binding (FIG. 2B).

Since VLA-4 also has extracellular matrix binding characteristics, modulations of migratory characteristics of B16.F10 cells by LysoPC were evaluated by the microscopic SACED cell migration assay on a fibronectin substrate (FIG. 8 A). The data clearly indicate that, in contrast to VLA-4 binding data, already short term LysoPC treated B16.F10 cells displayed a significantly reduced (p<0.05) motility regarding lamellipodia retraction velocity (FIG. 8 A-a). Additionally, frequencies of lamellipodia protrusion and retraction were significantly reduced (p<0.05) after 48 h of LysoPC treatment (FIG. 8 A-b).

P-selectin mediated interaction of tumor cells with platelets is pivotal for the metastatic process. Thus, we investigated whether LysoPC treatment of the B16.F10 cells also changes this metastasis-relevant interaction and found significantly reduced (p<0.001) P-selectin mediated interaction with activated platelets. Already a short term LysoPC treatment of B16.F10 cells decreased activated platelet binding to the level of non-activated platelets (p=0.748) (FIG. 8 B). Using a specific P-selectin blocking antibody vs. a control antibody, the P-selectin specificity of platelet interaction in control cells could be confirmed (p<0.05), while the significantly reduced binding by LysoPC-treatment in B16.F10 cells was not further (p=0.552) affected by the antibodies (FIG. 8 B).

Reduced expression levels of the adhesion receptors due to LysoPC might explain the decreased binding activities of VLA-4 and P-selectin in the LysoPC-treated cells. However, flow cytometry analysis using B16.F10 cells repeatedly incubated with 450 μM LysoPC or with DMEM for 13 days showed that LysoPC treatment did not influence VLA-4 receptor expression (FIG. 3A). Furthermore, the functionality of VLA-4 to interact with soluble VCAM-1-Fc chimera was only slightly impaired (FIG. 3B) and did not reflect the completely reduced binding as seen in the flow chamber assays (FIG. 2B). Furthermore, the expression of P-selectin ligands as detected by P-selectin-Fc chimera binding remained unchanged in LysoPC treated B16.F10 compared to control cells (FIG. 3C), in contrast to the total inhibition of cell binding to activated platelets, already after short term LysoPC treatment (FIG. 8 B).

Taken together, the unchanged expression of adhesion receptors and binding to soluble ligands might suggest that alterations of cell membrane characteristics by the LysoPC treatment may be responsible for the strikingly reduced interaction with cellular ligands. Such an assumption is strongly supported by SEM analysis comparing the LysoPC treated and untreated cells (FIG. 8 C). It became evident that LysoPC treatment induced a rapid increase in cell surface lamellipodia (FIG. 8 C-b) compared to untreated cells (FIG. 8 C-a).

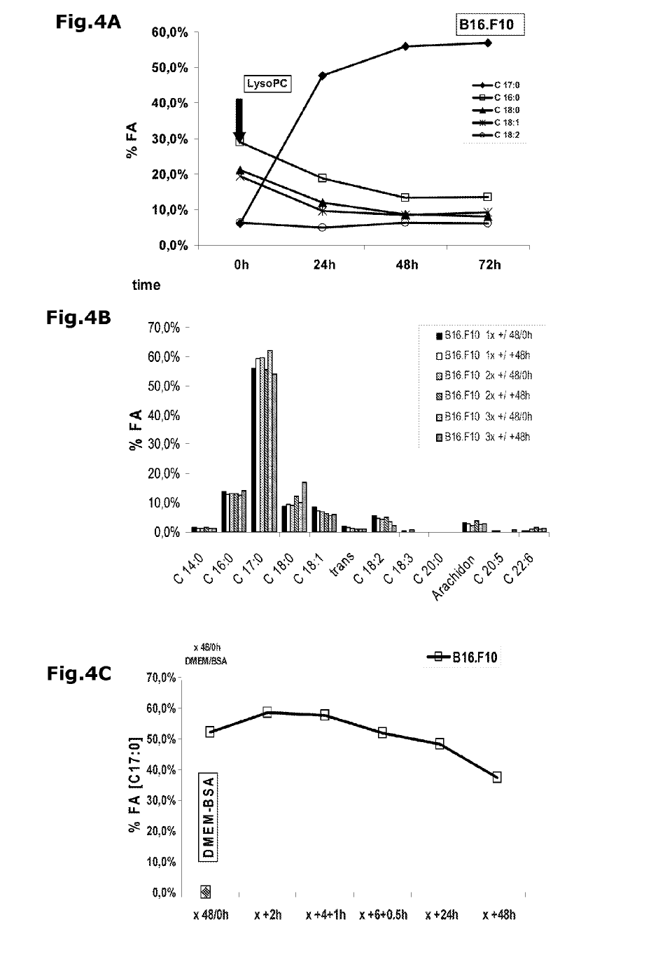

Example 3: Cell membrane alterations induced by LysoPC treatment. In order to analyze membrane alterations caused by exogenous LysoPC the kinetics of changed fatty acid profiles of B16.F10 cells were evaluated by GC analysis. Incubation of the cells with HEP-LysoPC resulted in a rapid increase of the ratio of C17:0 from 5% (0 h) to about 50% after 24 h (FIG. 4A). Between 48 h and 72 h, a plateau was apparently reached (C17:0: ˜55%). Ratios of all other investigated fatty acids decreased accordingly. Similar kinetics were observed using HEX-LysoPC, but with a baseline ratio of about 30% of total FA (data not shown). Repeated feeding (up to six times) every 48 h with LysoPC did not further change the membrane composition in treated B16.F10 melanoma cells (FIG. 4B).

In contrast to the rapid incorporation of the FA from exogenous LysoPC, the altered membrane composition was found to be longer lasting after replacing LysoPC containing medium by LysoPC-free culture medium. Even 48 h after incubation in LysoPC-free DMEM medium, the fatty acid ratio was reduced only by 10-15% (FIG. 4C). This indicates that the high membrane turnover occurs only in contact with external LysoPC.

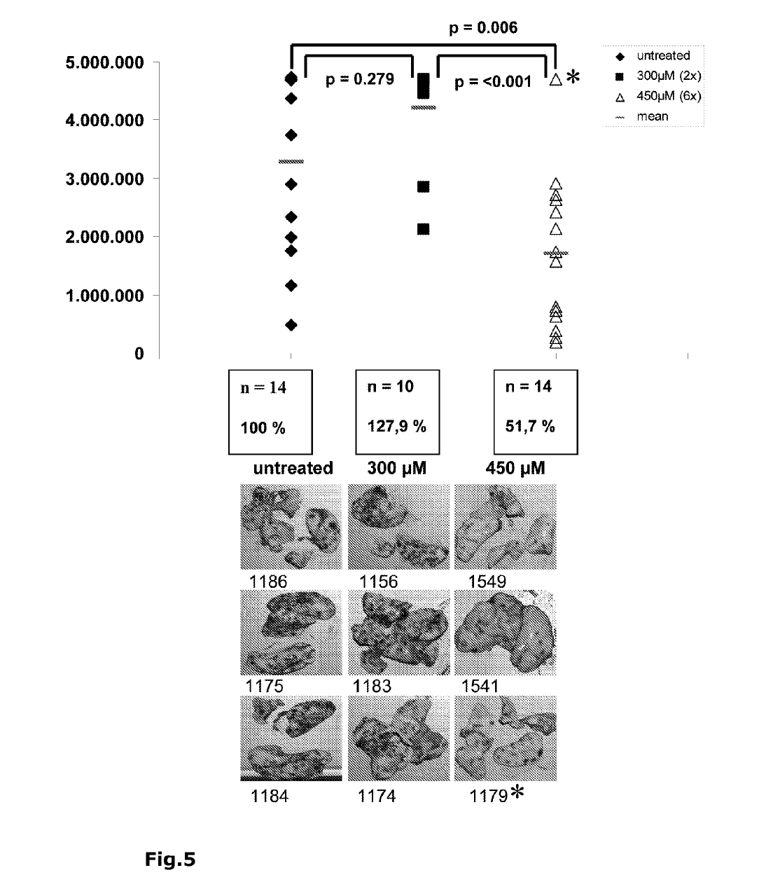

Example 4: Inhibition of in vivo metastasis of B16.F10 ELN cells pretreated with LysoPC. LysoPC-caused drop in cell adhesion via VLA-4, P-selectin or modified cell surface properties in vitro, could also refer to reduced B16.F10 cell adhesion in vivo and thus to a decreased metastatic rate. Therefore, we compared the metastatic spread of untreated B16.F10 cells with those cells treated twice with 300 μM LysoPC (C17:0), or cells treated six times with 450 μM LysoPC (FIG. 5). For a sensitive quantitative analysis of metastatic spread in the lung, luciferase-transduced B16.F10 ELN cells were used. This advanced metastasis model is described in more detail in the supplemental data section (FIG. 9).

Fourteen days after tumor cell injection, homogenized lung extracts of long-term B16.F10-450 μM group displayed a significantly reduced luciferase activity compared to the untreated control (p=0.006) but also to the short-term B16.F10-300 μM group (p=0.001). Comparing the control and the short-term 300 μM group, a slightly but insignificantly enhanced luciferase activity became obvious (FIG. 5). This could be caused by the lower standard deviation in the latter group (22.0% versus 45.5% of the mean in controls).

The luciferase data were clearly confirmed by macroscopically visible metastasis, as three representative examples of lung surfaces of each group demonstrate (FIG. 5, lower part). While no differences were seen between the control and the short-term group, clearly smaller numbers of metastatic foci were observed in the long-term 450 μM LysoPC group.

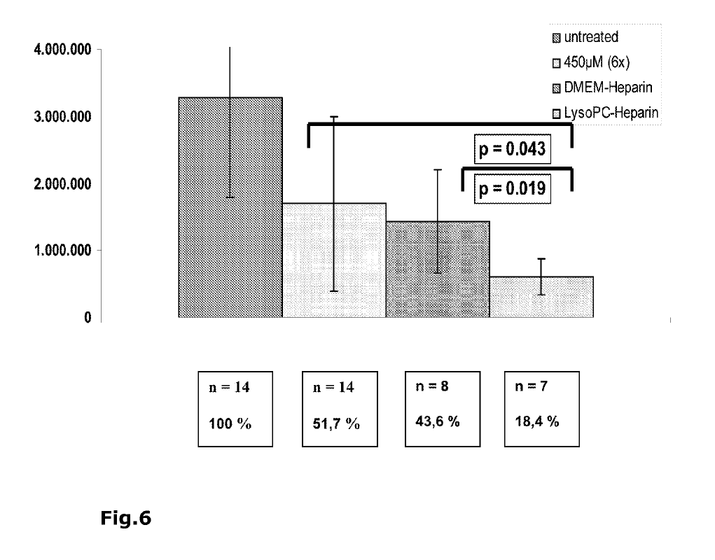

Example 5: Synergistic effect of long-term LysoPC pre-treatment of cells and heparinization of mice: Anti-metastatic effects of heparin are well documented referring to different molecular targets, among others inhibition of P- and L-selectin and VLA-4 (25-28). To compare the effects of long-term LysoPC pre-treatment of B16.F10 cells with intravenous heparin, we analyzed metastatic spread of control and 450 μM pre-treated B16.F10 cells in heparinized mice. As shown in FIG. 6, both long-term LysoPC treatment as well as heparin injection significantly reduced metastatic invasion compared to untreated control (51.7%, p=0.006 vs. 43.6%, p=0.004). Whereas the differences between both groups were not significant (p=0.603), the injection of LysoPC pre-treated cells into heparinized mice caused a synergistic effect, i.e. further reduced the occurrence of metastasis to 18.4%, which was significantly lower than by LysoPC (p=0.043) or heparin (p=0.019) alone.

Supplementary Data, Detailed Description of FIGS. 7 to 9

FIG. 7 shows the following: A) Effect of bovine serum albumine (BSA) on LysoPC-mediated toxicity. LysoPC was toxic towards B16.F10 cells (p<0.001) at physiological concentrations (≧150 μM) in culture medium (DMEM plus 10% FCS). Thus, different amounts of BSA were added to reduce the concentration of cellular assessable LysoPC (monomolecular and micellar dissolved) and to therefore reduce LysoPC toxicity. Viability of the tumor cells was determined by BrdU assay and adding 10 mg BSA/ml, reduced cell viability was evident above 300 μM LysoPC. Using 20 mg/ml, reduced cell viability was evident above 450 μM LysoPC. Thus, to avoid toxicity of exogenous LysoPC (150 μM to 900 μM) towards the tumor cells during the LysoPC-removal-assay (FIG. 1), addition of 20 mg-40 mg BSA/ml DMEM was essential.

B, C and D) Colorimetric assays for the quantification of LysoPC removal from the supernatant of B16.F10. The removal of the added LysoPC from the supernatant of B16.F10 cells was determined by a combination of two colorimetric assays. The first assay—a commercial PL assay (mti diagnostics GmbH)—detects choline bound to LysoPC plus free choline (B). The assay is based on the enzymatic liberation of choline from PC-containing PLs by a phospholipase D activity followed by quantification of free choline by a choline oxidase activity coupled with a common peroxidase reaction (oxidation of choline resulted in betaine plus hydrogen peroxide, which oxidizes 4-aminontipyrine and phenol to a chromogen with a maximum absorption at λ=505 nm) (Chap, H. H. et al., Clin. Chem. 34:106-109 (1988); Kishimoto, T. et al., Clin. Biochem. 35:411-416 (2002)).

In contrast, the second assay only quantifies free choline (FC) (C). The second FC assay is identical to the first one, but lacking phospholipase D. It was especially produced for this purpose by mti diagnostics GmbH.

Since both, LysoPC and free choline were detected by the PL assay (D: black curve) and because the removal of LysoPC by B16.F10 cells is accompanied by the generation of free choline (D: red curve) both assays were used to determine the actual removal of LysoPC. Subtracting the values of free choline (FC assay) from those of free choline plus LysoPC (PL assay) results in the actual LysoPC values (D: blue curve). HPTLC-analysis showed that only traces of other PC-containing phospholipids (from FCS) were present during the LysoPC removal experiments (not shown), which is a prerequisite to use these assays in combination for LysoPC-quantification.

The assays were performed in microtiter plate volumes with 50 μl culture supernatant or cell lysate and 200 μl detection reagent. The specificity of both assays was shown by using increasing concentrations (0-3,600 μM) of free choline, HEX-LysoPC, HEP-LysoPC as well as two other possible LysoPC degradation products, glycerophosphocholine (GlyceroPC) and phosphocholine. While phosphocholine was not detectable in both assays, the assay containing phospholipase D was able to quantify LysoPC, GlyceroPC and free choline, while the second assay was only able to quantify free choline. Linearity for all choline species is given between 0 and 2.00E±03 μM.

The assay combination could be successfully used to quantify the removal of exogenous LysoPC from the supernatant of B16.F10 cells. Removal of LysoPC from the supernatants is clearly accompanied by an emergence of free choline, which is independent of the LysoPC species (C16:0, C17:0), the LysoPC concentration (ranging from 300 to 900 μM) and the amounts of BSA added (20 or 40 mg/ml) (data not shown).

FIG. 8 shows the impact of short-term exogenous LysoPC treatment of B16.F10 cells on P-selectin-mediated platelet binding, morphology and migratory properties on a fibronectin substrate. A) Motility data of B16.F10 cells on fibronectin layers derived by SACED assay displayed significant (p<0.05) differences in migration velocities. a) B16.F10 cells incubated with 450 μM LysoPC for 24 h showed a slower lamellipodia retraction velocity than cells without LysoPC. b) Analysis of lamellipodia dynamics (i.e. frequencies) revealed attenuated dynamics (p<0.05) of lamellipodia protrusion and retraction regarding the cells incubated with 450 μM LysoPC for 24 h.

B) Binding of calcein-labeled TRAP-14 activated platelets to B16.F10 cells treated with 450 μM LysoPC for 48 h was analyzed by fluorescence microscopy. Untreated B16.F10 cells displayed a strong interaction with activated platelets, which could be significantly (p<0.05) inhibited by a blocking P-selectin mAb, but not by non-specific control antibodies. Only background binding was observed with resting platelets. LysoPC treatment of B16.F10 cells significantly (p<0.001) reduced P-selectin mediated interaction with activated platelets to the level of resting cells. Data represent means±SD from three independent experiments. P, resting platelets; aP, activated platelets; LysoPC, LysoPC treated B16.F10 cells; Ab, Ig-matched control antibody.

C) Cell surface alterations with respect to LysoPC treatment of B16.F10 cells were determined by scanning electron microscopy (SEM). Images of B16.F10 cells grown on glass slides and coated with platinum/palladium were taken a) without LysoPC treatment or b) after LysoPC incubation. While only a few lamellipodia were visible in untreated cells, an augmented number of lamellipodia was recognizable after LysoPC treatment. (bars=20 μm, image sections correspond to a=5 μm and b=2 μm).

FIG. 9 shows the detection of metastatic lesions by luciferase-transduced B16.F10 melanoma cells in the lung and other tissues. To establish the mice metastasis model with luciferase-transduced B16.F10 melanoma cells, metastatic lesions in the lung and other tissues (those strongly supplied with blood, spleen, liver, kidney) were investigated at different time points (6 h, 24 h, 7 d, 14 d, and 19 d). Sensitivity of in vitro detection of metastatic lesions was determined as recently described (Jantscheff, P. et al., Prostate 69:1151-1163 (2009)) and limited corresponding to the detection of about 1000 cells in 1 ml tissue lysate (data not shown). Three to four animals were analyzed at each time point and metastatic lesions in lung and other tissues were determined by in vitro luciferase assay in tissue homogenates (see Material and Methods). As shown in A) significant metastasis could be detected first at day 7 and reached a plateau at day 14 in the lung. The experiment was terminated at day 19 since general health situation of the animals was critical, caused by the strongly infiltrated lungs. At this time point dilution of lung extracts did not change luciferase activity (up to 1:100) whereas at day 14 a dose dependent reduction was seen diluting lung extracts from 1-1:100 (not shown). Therefore, in future experiments the animals were analyzed at day 14 and the experiments were limited to this time frame. Using the same scale, no metastatic lesions were detected in the other organs. However, as shown in B (day 14) and C (day 19) metastatic spread was also observed in the other tissues of individual mice, but to an at least 1,000 times lesser extent than in the lung. Metastatic spread in these organs showed a wide range between individual mice as well as within individual tissues (A, B). The relatively low invasion into these tissues made it irrelevant to follow up these metastases in the present study.

Metastatic spread was also measured once weekly by in vivo bioluminescence as described previously (Jantscheff, P. et al., Prostate 69:1151-1163 (2009)). Overlays of in vivo images and mouse photographs allowed to localize the metastatic lesions in lung and at the injection sites (D: black arrows) even in late images (D: day 14). But in contrast to former studies (Jantscheff, P. et al., Prostate 69:1151-1163 (2009); Jantscheff, P. et al., submitted 2009; Janthscheff, P. et al., Clin. Exp. Metastatsis: in press (2009)), quantification of LysoPC-treated and untreated animals could not confirm (data not shown) the differences observed by in vitro analysis or macroscopic images (FIG. 5), ratifying recent data of Niers et al. (Niers, T. M. et al., Clin. Exp. Metastasis 26:171-178 (2009)) in the same model.

1. A medicament for treating or preventing metastatic spread of tumors comprising a phospholipid component and a polysaccharide component having polysaccharides containing sulfate groups.

2. The medicament of claim 1, wherein the phospholipid component comprises at least one phospholipid containing ω-3-fatty acids and/or containing no or only low amounts of ω-6 fatty acids.

3. The medicament of claim 2, wherein
(i) in the phospholipid component said at least one phospholipid is selected from 1,2-diacylglycerophospholipids, 1-acylglycerophospholipids and 2-acylglycerophospholipids having saturated and/or unsaturated acyl residues and pharmacologically acceptable salts thereof; and/or
(ii) in the phospholipid component said at least one phospholipid contains acyl residues selected from saturated acyl residues, ω-3- and ω-9-fatty acid residues and mixtures thereof; and/or
(iii) in the phospholipid component said at least one phospholipid is a phosphatidylcholin; and/or
(iv) in the phospholipid component said at least one phospholipid contains no or only low amounts of ω-6 fatty acids and the phospholipid component further comprises an oil having a high content of ω-3-fatty acids; and/or
(v) in the phospholipid component said at least one phospholipid is a phospholipid of marine origin (MPL); and/or
(vi) in the phospholipid component said at least one phospholipid is a hydrogenated phospholipid containing essentially saturated fatty acids; and/or
(vii) in the phospholipid component said at least one phospholipid is a milk derived phospholipid having no or only low amounts of ω-6 fatty acids and containing high amounts of ω-9 fatty acids.

4. The medicament of claim 1, wherein
(i) the phospholipid component further contains triglycerides, free fatty acids, diglycerides and/or monoglycerides, wherein the total content of said further glycerides and fatty acids is no more than 90% by weight of the total lipids of the component; and/or
(ii) the phospholipid component contains oils.

5. The medicament claim 1, wherein said phospholipid component comprises at least one phospholipid compound represented by the formula I 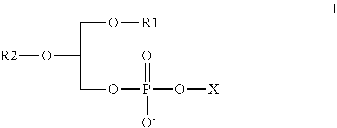
wherein
R¹ and R² are independently selected from H and C₁₆₋₂₄ acyl residues, which may be saturated or unsaturated and may carry 1 to 3 residues R³ and wherein one or more of the C-atoms may be substituted by O or NR⁴;
X is selected from H, —(CH₂)ₙ—N(R⁴)₃⁺, —(CH₂)ₙ—CH(N(R⁴)₃⁺)—COO⁻, —(CH₂)ₙ—-CH(OH)—CH₂OH and —CH₂(CHOH)ₙ—CH₂OH, wherein n is an integer from 1 to 5;
R³ is independently selected from H, lower alkyl, F, Cl, CN and OH; and
R⁴ is independently selected from H, CH₃ and CH₂CH₃, or a pharmacologically acceptable salt thereof.

6. The medicament of claim 5, wherein R¹ and R² are independently selected from H and unsubstituted C₁₆₋₂₄ acyl residues, which may be saturated or unsaturated, and X is selected from a choline, serine, ethanolamine, glycerol and inositol residue.

7. The medicament of claim 6, wherein
(i) at least one of R¹ and R² is an ω-3-fatty acid residue having at least 20 C-atoms or is an ω-9-fatty acid residue having at least 18 C-atoms; and/or
(ii) the content of ω-3- and ω-9-fatty acid residues is at least 20% by weight of all acyl residues of the phospholipid component; and/or
(iii) the weight ratio of ω-3-fatty acids and/or ω-9-fatty acids to ω-6-fatty acids in the acyl residues of the phospholipid component is at least 10:1; and/or
(iv) R¹ and R² are independently selected from H and unsubstituted and saturated C₁₆-₂₄ acyl residues; and/or
(v) R¹ and R² are independently selected from H and unsubstituted ω-9-C₁₆₋₂₄ alkenoyl residues.

8. The medicament of claim 6, wherein the at least one phospholipid is selected from oleoyl-, palmitoyl-, heptadecanoyl-, and stearoyl-glycerophosphocholines and the respective lyso compounds.

9. The medicament of claim 1, wherein the polysaccharide component comprises at least one component selected from heparins.

10. The medicament claim 1, wherein
(i) the phospholipid component and the polysaccharide component are present in two separate preparations; or
(ii) the phospholipid component and the polysaccharide component are present in one single preparation.

11. The medicament of claim 10, wherein the phospholipid component is for intravenous, subcutaneous, intracutaneous or intraperitonal application and comprises lysophospholipids.

12. The method of claim 16, wherein the tumors are solid tumors.

13. (canceled)

14. The method of claim 12, wherein the solid tumors are selected from melanoma, pancreascarzinoma, prostatacarzinoma, ovarialcarzinoma, and mammacarzinoma.

15. The method of claim 12, wherein the phospholipid component and the coagulation modulating component are present in
(i) two separate preparations, or
(ii) in one single preparation.

16. A method for inhibiting metastatic spread of tumors, which comprises contemporaneously or subsequently administering a patient in need of such treatment a medicament of claim 1.
